(19) United States
(12) Patent Application Publication
(10) Pub. No.: US 2023/0052976 A1
(43) Pub. Date: Feb. 16, 2023

(54) Method for fermenting biomass and producing material sheets and suspensions thereof

(71) Applicant: MAKEGROWLAB Sp. z o.o., PULAWY (PL)

(72) Inventors: MAGDALENA RÓZA BRITO, PULAWY (PL); ROBERT JOSHUA BRITO, PULAWY (PL)

(21) Appl. No.: 17/589,198

(22) Filed: Jan. 31, 2022

Related U.S. Application Data
(60) Provisional application No. 63/227,236, filed on Jul. 29, 2021.

(51) Int. Cl. C12P 19/04; D21H 25/02; D21H 11/20

(52) U.S. Cl. CPC C12P 19/04; D21H 25/02; D21H 11/20; C12P 2201/00; C12P 2203/00

(57) ABSTRACT

A method is described to produce cellulose sheets and suspensions by fermenting biomass obtained from household and/or industrial waste. The inoculum in the fermentation includes cellulose producing bacteria and optionally yeast cells. The method has a high cellulose productivity. The resulting sheets or suspensions can be used to produce various further materials, such as disposable vessels, sachets, artificial leather. The sheet and suspensions can be used as additives in material production, such as paper making production. The method provides an alternative to make disposable items that are currently made of plastic, and textiles.

### PRIORITY

This application claims priority from a U.S. provisional application No. 63/227,236 filed on Jul. 29, 2021, the contents of which is incorporated herein by reference.

### FIELD OF THE INVENTION

This invention relates generally to production of biodegradable materials and reuse of biowaste material from industry and households. More specifically the invention relates to a process of fermenting biomass and using the fermented biomass for production of material sheets and their suspensions. The invention further relates to production of biodegradable materials for manufacturing utility items, packing materials, and artificial leather among other uses.

### BACKGROUND OF THE INVENTION

Biomass, as meant here, is organic matter originating as waste from industry as well as from households. Biomass may include plant or animal residues and may be obtained from households, agriculture, industrial plants, forestry, maintenance of public green areas, food production plants and so on. Examples of biomass sources are including but not limited to fruits, vegetables, grains, residues of plants and agriculture waste or fluids from the food and beverage sector which can be seen as leftovers or waste.

As part of efforts to decouple energy needs from fossil fuels, there has also been a trend towards advanced biomass conversion, mainly for electricity generation in thermal power plants. On the other hand, there are continuous efforts to produce biodegradable materials for example for use in textile industry, papermaking industry, or the food packaging industry to mention a few.

Biodegradable materials and products made of biomass could also be suitable in water purification e.g., in absorption of toxins and petroleum wastewater, in acoustics, such as speaker membranes. Biodegradable materials and products made of biomass would be an excellent alternative to plastic products as well.

Bacterial cellulose fibers have been disclosed to be produced in flow-through bioreactors e.g., in U.S. Pat. No. 5,846,213 and CA 2,207,988. On the other hand, a two-stage process for culturing bacterial cellulose is described e.g., in US 2006/2347 and WO2004/50986 in a two-stage process ensuring production of a film having a grammage of 10-45 g/m².

Polish patent PL412146 describes a method for the production of bacterial cellulose consisting in the preparation of an inoculum by inoculating on Herstin-Schramm medium containing glucose, yeast extract, bacterial peptone, citric acid, Na2HPO4, MgSO4.H2O, sterilized and enriched with ethyl alcohol, Gluconacetobacter xylinus bacteria stored on a medium with the following composition: yeast extract, bacterial peptone, citric acid, Na2HPO4, MgSO4.7H2O, bacteriological agar, sterilized and enriched with ethyl alcohol, then mixed for 15 minutes and incubated for 7 days at 28-30° C., re-mix the culture for 5 minutes and transfer the inoculum obtained in this way on fresh Herstin-Schramm medium with the same composition for production culture. The method is characterized by the fact that the production culture is carried out in the presence of a rotating magnetic field with a rotational frequency in the range of 10-50 Hz, magnetic induction in the range of 5-35 mT, for 3 days, with a pH in the range 4.5-5.5, at a temperature of 28-30° C. The obtained cellulose membranes are transferred to clean containers, washed with distilled water, and then purified by incubation in 0.1 M aqueous NaOH at 90° C. for 30 minutes, the purification procedure being repeated three times, then the purified cellulose is rinsed in water distilled until the pH stabilizes at 6.5-7.5 and dried at 60° C. until constant weight is obtained.

Polish patent publication PL212003 describes a method of obtaining bacterial cellulose in stationary culture condition using Acetobacter xylinum-strain. Before actual production culture, the entire volume of the medium was preincubated under stationary conditions for 24 hours at 27-33° C. and then after thorough mixing of the medium and pouring into bioreactors in such amounts that the surface to volume ratio was 0.6-0.8 cm⁻¹ proper production culture was carried out in stationary conditions for 5-7 days.

Korean publication KR20170013443 relates to a device for collecting the bacterial medium of cellulose culture and a method for producing bacterial cellulose using this device. The device includes multiple cultivation trays arranged in multiple layers of cultivated microorganisms, a hydraulic unit to position the inclined cultivation trays, a fluid collection line, connected to each of the cultivation trays and a collection medium, a filter to remove contaminants in the cultivation medium, in which the liquid collection line is integrated and connected as one; a culture tank for storing and culturing the medium which is connected to a filter, a vacuum pump for transferring the culture medium connected to the culture tank, and a device for regulating the collection of liquid, controlling the collection and feeding of the culture medium. The apparatus collects the culture medium discarded after the cultivation and feeds the sterilized culture medium to the harvested culture medium. As a result, the bacterial cellulose can be grown continuously without a pre-culturing step.

Accordingly, there are methods disclosed to provide bacterial cellulose production in processes where the starting material is for example a glucose medium inoculated with cellulose forming bacterial inoculate. However, the pressing issue of today's reality is not only how to make biodegradable material, but also how to reuse waste. Therefore, there is a need for methods to produce biodegradable material in a process where the starting material is a low-cost waste material e.g., any kind of household or industrial waste materials. The present disclosure provides such methods.

Bacterial cellulose is commonly produced by Hestri-Stramm (HS)-medium consisting of 2.0% glucose (w/v), 0.5% yeast extract (w/v), 0.5% peptone, 0.27% Na2HPO4 (w/v), and 0.15% citric acid (v/v) HS medium is generally considered expensive for production of materials to implement in single use packaging or textiles. Accordingly, there is a need for more economic mediums for producing bacterial cellulose for producing materials for various purposes.

In other known applications non continuous, static fermentation processes are used. For example a food product called Nata de Coco is produced by fermenting coconut water that gels by production of cellulose producing bacterial Komagataeibacter xylinus. The process requires high manual labor by adding the medium, mixing medium with inoculum and preparing single portions of inoculum for non-continuous static process.

A common practice in production of bacterial cellulose is the use of single bacteria colonies to produce bacterial cellulose. The drawback of using single bacteria colonies is that they are highly sensitive to their surrounding environment, making them highly prone to contamination, and substantial financial loss. Production facilities using single colony bacteria require high capital to ensure sterile conditions. This is one of the reasons for high costs of bacteria derived cellulose and explains common use of this cellulose mainly for medical, pharmaceutical or cosmetic fields. Due to the high cost, packaging and textile industries do not generally use bacterial cellulose as their raw material.

Accordingly, there is a need for more economic practices. There is a need for practices that do not require contamination free surroundings, or high labor. There is a need for economic production of bacterial cellulose in continuous production systems.

### SUMMARY OF THE INVENTION

The invention disclosed here provides solutions to the above-mentioned problems and addresses other issues as well.

It is an object of this invention to provide a method to use of organic waste based mediums with lower cost, additionally different waste sources; exemplary sources are wastes from fruit and vegetable juice production e.g. apples or carrots, winery waste, brewery waste, coconut water waste, algae, sugar production waste, oat milk and other vegetable milk production, city maintenance waste, e.g., grass, which do not limit the invention to be used in different geographical zones and seasons.

It is an object of this invention to provide a method where process of production of bacterial cellulose is adjusted for industrial purposes without limitation on certain regions due to different waste sources and low manual work due to continuous fermentation method with high yields and piping connections between process steps that enable industrial automations resulting in minimizing labor.

It is an object of this invention to provide a process with use of symbiotic colonies of bacteria and yeasts.

It is an object of this invention to provide a method to use symbiotic cultures that decrease contamination risk while maintaining predictable yields of cellulose production.

A further object of this invention is to provide a method resulting in higher yields than single production with single bacteria colonies.

It is a further object of this invention to provide a method that allows obtaining yields in shorter time than method using only a single bacteria colonies and the yields are obtained in shorter time than using wild scoby (symbiotic culture of bacteria and yeast) used in kombucha fermentation.

It is an object of this invention to provide a method to produce cellulose containing biodegradable materials from household or industry waste, the method comprising:
a. obtaining biomass derived from the waste;
b. mixing the biomass with water and raising the temperature to at least 120° C.;
c. obtaining a filtered primary raw material by filtering the biomass;
d. directing the filtered primary raw material into one or more vessels;
e. supplementing the filtered primary raw material in the one or more vessels with one or more of ammonium salt of sulfuric acid, calcium, and magnesium; and adding an inoculum suspension comprising cellulose producing bacteria;
f. adjusting pH of the supplemented primary raw material in the one or more vessels to below pH 7, and sugar level of the material to 5-15 w-%;
g. obtaining a main mixture by allowing the supplemented primary raw material from step f) to ferment in the one or more vessels at 22-33° C.;
h. dividing part of the main mixture into containers and incubating the main mixture in the containers at 22-33° C.;
i. adding portions of the main mixture incrementally from the one or more vessels to the containers every 4-15 days throughout the incubation period of step h);
j. Obtaining a raw material from the containers by draining the main mixture form the containers and collecting the raw material into a collection container and directing the drained liquid back to the one or more vessels;
k. obtaining purified raw material by cleaning the raw material in the collection container; and
l. drying the purified raw material on a substrate to form dry cellulosic sheets.

In the method the cellulose producing bacteria is selected from the group consisting of Acetobacter sp., Glucobacter s.p, Sarcina sp., and Komagataeibacter sp.

In the method the inoculum suspension comprises additionally yeast cells. The inoculum comprises more bacterial cells than yeast cells.

In the method the inoculum suspension may comprise a symbiotic culture of bacteria and yeast, or Saccharomyces cerevisiae cells.

It is an object of the invention to provide a method to produce cellulose containing biodegradable materials from household or industry waste, wherein efficiency of production of bacterial cellulose is 50-950 g/l of the main mixture. Efficiency of production of dry sheets is 5-90 g/l of the main mixture.

It is an object of the invention to provide a method to produce cellulose containing biodegradable materials from household or industry waste, wherein the method further comprises a step of blending the raw material or the purified raw material to obtain a suspension. According to certain aspects the dry sheets may be grinded and added to the suspension.

It is an object of the invention to provide a method to produce cellulose containing biodegradable materials from household or industry waste, wherein the dry sheets are further treated to form sachets, disposable vessels, or artificial leather.

An object of the invention is to provide cellulosic dry sheets made by a method disclosed here.

An object of the invention is to provide cellulosic suspension made by a method disclosed here. The suspension is suitable as an additive to paper and acts as an air barrier.

### BRIEF DESCRIPTION OF THE DRAWINGS

FIG. 1 is a schematic representation of an example of the method as is described in Example 3.

FIG. 2 is a schematic representation of an example of the method as is described in Example 4.

FIG. 3 is a schematic representation of an example of the method as is described in Example 5.

FIG. 4. is a schematic representation of an example of the method as is described in Example 6.

FIG. 5 shows disposable single use vessel described in invention.

FIG. 6 shows pouches made by method described in invention.

FIG. 7 shows leather alternative textile made by method described in invention.

FIG. 8 Shows suspension made by using grinding dried sheets.

FIG. 9 shows dry material made from sheet previously grinded.

FIG. 10. shows a small-scale example of production of Bacterial cellulose on 100 ml HS-medium or apple waste medium of this invention cultivated on 10% Inoculum of single or symbiotic culture with or without addition of CaCl2 as Ca²⁺ ions source. It can be seen that the single strain bacterial cultures produced clearly less than the symbiotic cultures, and that CaCl2 addition improved the production of bacterial cellulose.

source.

### DETAILED DESCRIPTION OF THE INVENTION

As used here, the term “Biomass” is material that is obtained from byproducts of food and agriculture production and/or at least partially decomposed household or industrial biowaste optionally by pressing fluids out of the decomposed waste. Non-limiting examples of waste suitable to be used in the method disclosed here are any kind of food waste, agricultural waste, waste from winery or brewery industries.

It is generally known that cellulose producing bacteria are efficient in producing cellulose fibers when the growth medium comprises abundant carbon and minimal amounts of nitrogen. Therefore, all the known methods to produce cellulose fibers include growing the bacteria on a glucose medium. Industrial and household waste on the other hand are known to comprise large amounts of nitrogen. Additionally, waste material comprises various metabolites that may not be optimal for bacterial cellulose production. For these reasons, household or industrial waste material has generally not been considered a suitable growth medium for cellulose producing bacteria.

Surprisingly here, a method has been developed which is suitable for cellulose producing bacteria to grow and produce high yields of bacterial cellulose material. The method is suitable for production of bacterial cellulose by using cellulose producing bacteria alone or in combination with yeast. The method disclosed here does not require isolating the cellulose fibers but biodegradable material can be produced without the isolation process.

A method is disclosed where biodegradable cellulosic material can be produced in a continuous process by cultivating the bacteria or the bacteria and yeast on pretreated biomass and biodegradable cellulosic sheets are provided suitable for use in making various materials, such as packing material, textiles, etc.

The invention is now described with reference to the non-limiting drawings and examples appended herein. A method to produce cellulose containing biodegradable materials from household- or industrial bio waste is described here.

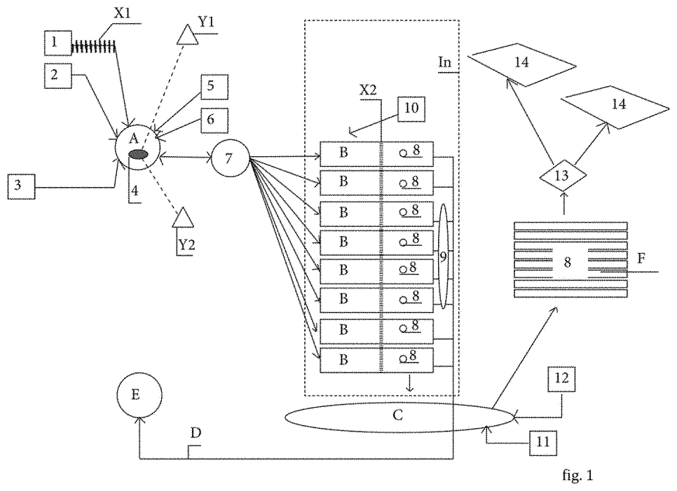

Referring to FIG. 1 and example 3, in the first stage a substantially dry biomass 1 is obtained. If the biomass originally contains fluids, the fluids are pressed, squeezed, centrifugated or by other mechanical manner removed from the biomass. The waste may be partially decomposed. Exemplary sources are wastes from fruit and vegetable juice production e.g. apples or carrots, winery waste, brewery waste, coconut water waste, algae, sugar production waste, oat milk and other vegetable milk production, city maintenance waste, e.g, grass, which do not limit the invention to be used in different geographical zones and seasons.

The obtained substantially dry biomass is then mixed with water 1 to 200 g/l, preferably 1 to 100 g/l, more preferably 5-50 g/l. According to certain aspects 10 g of substantially dry biomass is mixed with liter water. The temperature is raised to at least 120° C. for 10 to 150 minutes, more preferably 15 minutes to 2 hours. Preferably the temperature is kept within a range of 90 C-120° C. for at least 15 minutes.

After the step of heating the water-biomass mixture, this mixture is filtered using filter material (e.g. a mesh) (X1) having holes with size of 0.1-5 mm and more preferably 0.2-2 mm. The resulting filtered primary raw material has particle size of 0.1-5 mm and preferably 0.2-2 mm. The particle size can also be 0.5-1 mm.

The filtered primary raw material is then directed to one or more vessels A, where the process of mixing the filtered primary raw material with an inoculum 3 comprising cellulose producing bacteria takes place. In the vessel(s) the filtered primary raw material may be supplemented with ammonium salt of sulfuric acid (NH₄) 2SO₄ in an amount of 1-5 g/l of the filtered primary material. The filtered primary material may be supplemented alternatively with calcium 0.005 g/l to 1 g/l, preferably 0.05-0.5 g/l, more preferably 0.1-0.2 g/l. In certain aspects 0.15 g/l of calcium is supplemented to the primary material. Alternatively, or additionally magnesium may be supplemented into the primary material in an amount of 0.007-2 g/l, preferably 0.1-0.5 g/l, most preferably 0.2 g/l of the filtered primary raw material.

Inoculum of active strains of producing cellulose or strains producing cellulose in symbiosis with yeast is added to the vessel(s) in an amount of 10-50% (w/w), preferably 20-40%, more preferably 25-35% and most preferably 30% of the filtered primary raw material.

The cellulose producing bacteria may be selected from the group consisting of Acetobacter sp., especially Acetobacter xylinum, Acetobacter hansenii and Acetobacter pasteurianus, Gluconobacter sp, Sarcina sp, Komagataeibacter rhaeticus.

The yeast source is obtained from wild SCOBY cultures from kombucha fermentation and/or Saccharomyces cerevisiae species.

Typically, the inoculum comprising cellulose producing bacteria and yeast contain scoby-source alone from kombucha fermentation, or scoby-source and cellulose producing bacteria. The proportion of scoby-source to cellulose producing bacteria can be within a range of 30:1 to 1:1. According to certain aspects the proportion is 29:1; according to some other aspects the proportion is in a range of 5:1 and 1:1. According to certain aspects the proportion is 2:1.

According to certain aspects the inoculum is a mixture of cellulose producing bacteria and Saccharomyces cerevisiae yeast. Most preferably the inoculum comprises more than 50% bacteria populations, thus exceeding the yeast cell population. The bacterial cells may be of one strain or there may be multiple strains in the inoculum. According to certain aspects the inoculum comprises strains of Komagataeibacter rhaeticus and Acetobacter xylinum.

Again, referring to FIG. 1, pH of the solution formed as a result of mixing the materials in the vessel(s) is adjusted to below 7, preferably between 2 and 7, and most preferably between 3 and 4.6. Adjustment 5 is preferably conducted by adding acetic acid CH₃CHOOH, but also other weak acids may be used.

Sugar level of the pH-adjusted solution is measured for example with a refractometer and if below 5% the sugar level is adjusted by adding liquid glucose 6 to reach sugar level of 5-14%, more preferably 5-10% and most preferably 9%.

The solution having pH and sugar level adjusted is allowed now to ferment in the vessel(s) at a temperature between 22-33° C. for a period of days to several weeks.

The resulting solution is here called the main mixture 4. The main mixture is now poured into containers B that have a height from 2 to 50 cm preferably 5 cm and it has surface size from 10 cm² to 1000 cm² preferably 200 cm′. Other dimensions may also be used as long as the height: surface size ratio is between 1:10-1:4.

Part of the main mixture is left in the one or more vessel(s) A where the temperature is kept between 22-33° C., preferably 27-30° C. for a period not exceeding 30 days. This main mixture portion left in the one or more vessels A can be used as inoculant in a subsequent round, i.e., as inoculant to be mixed with filtered primary raw material in one or more vessels. This way the process may be kept continuous.

The main mixture in the containers B is incubated at a temperature of 22-33° C., preferably 29° C. At least part of the main mixture in the one or more vessels A is used to be incrementally added to the containers every 3-15 days, preferably every 4 day throughout the incubation period that vary from 3 to 30 days. The added solution may be 1-70% preferably 1-10%, most preferably 5% of the volume of each container.

At the end of the incubation period a raw material is obtained from the containers by draining the main mixture from the containers preferably through a sieve. The raw material is collected into a collection container C and the drained liquid is directed via a pipe D back to the one or more vessels (A, E) so as to be used as part of inoculum in the next fermentation process.

The raw material in the collection container C is then cleaned to obtain purified raw material. The cleaning is preferably conducted by adding sodium hydroxide in an amount of 0.1-2% (w/w).

The purified raw material is treated with glycerin after which the material is dried on a substrate at 25-100° C. preferably 50-70° C.

According to certain aspects the raw material and/or the purified raw material is blended to obtain a suspension which is spread on the substrate. Glycerin treatment may be made before or after drying.

According to certain aspects the dried sheets may be grinded to make a suspension. FIG. 8 shows suspension made this way.

The substrate F may be a flat smooth surface preferably made of PVC, plexiglas, or silicone, but other materials are also possible. According to certain aspects the substrate may be a mesh. According to certain aspects the substrate is a textile mesh coated with fluoropolymers, such as polytetrafluoroethylene (Teflon™), however, other coating materials may also be used. The substrate F may be a flat surface. The substrate may have a 3D shape without negative angles. The substrate may comprise holes or protrusions have a diameter maximally of 20 mm, and the holes or protrusions being space at a distance from each other maximally at a distance of 20 mm.

The glycerin treatment may be by spraying, soaking or adding to suspension. Glycerin may be applied in amount of up to 8%, preferably between 0.2-5%, most preferably 0.5 to 1% of dry mass of cellulose.

The dried material formed on the substrate is collected and cut into sheets.

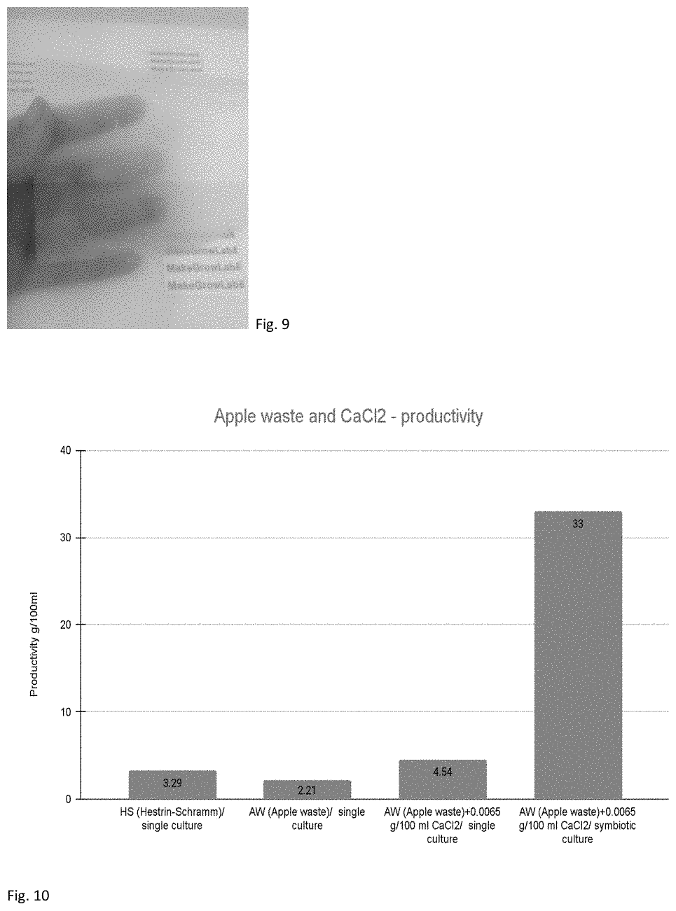

According to certain aspects of the invention, efficiency of the production of bacterial cellulose raw material is between 50-950 g/l of the main mixture. FIG. 10 shows an example of a small-scale experiment producing 33 g/100 ml. Example 4 below shows a pilot scale experiment where the efficiency was 4200 g/4500 l of main mixture.

According to certain aspects of the invention dry sheet efficiency is between 5-90 g/l of main mixture. On average the dry sheet efficiency is 50 g/l of main mixture.

The sheets are further subjected to processing to produce various useful materials, for example disposable vessels, sachets, textiles, packaging materials etc. These materials can further be used to make other products, such as pots, bags, biofiltration material.

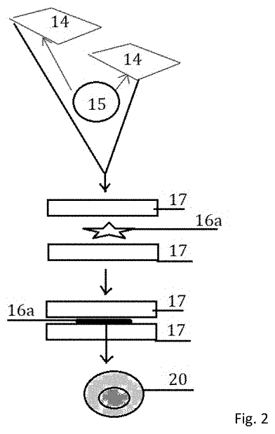

In reference to FIG. 2 and example 4, in order to produce a disposable vessel 20, the dry sheet 14 is moistened 15 to a minimum of 10% water content, and then the resulting blank 16a is placed in a press 17 with a pressure of at least 5 ton at a temperature of up to 125 C.°. As a result, a disposable vessel 20 of the desired shape is removed from the press.

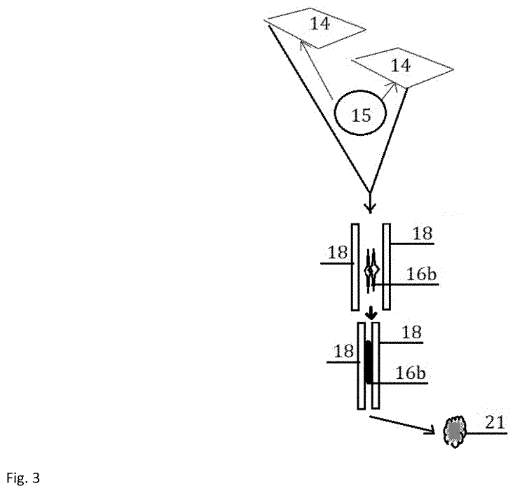

In reference to FIG. 3 and example 5, in order to produce a sachet 21, the dry sheet 14 is moistened 15 to a minimum of 10% water content, and then the blank 16b is placed in the foil welding device 18. In the final phase, the sachet 21 of the desired shape is withdrawn from the foil sealing device.

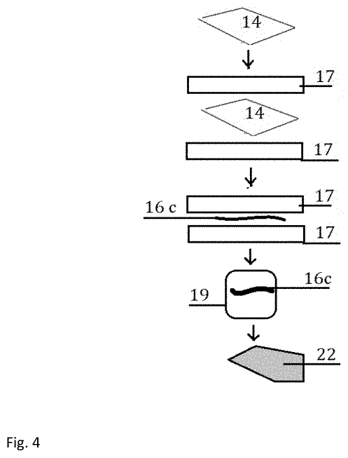

In reference to FIG. 4 and example 6, in order to obtain artificial leather 22, the dry sheet 14 is placed in a press 17 with a pressure of at least 5 tons at a temperature of up to 125 C.°, and then the resulting semi-finished product (blank) 16c is impregnated with a mixture of guar gum and natural resins 19.

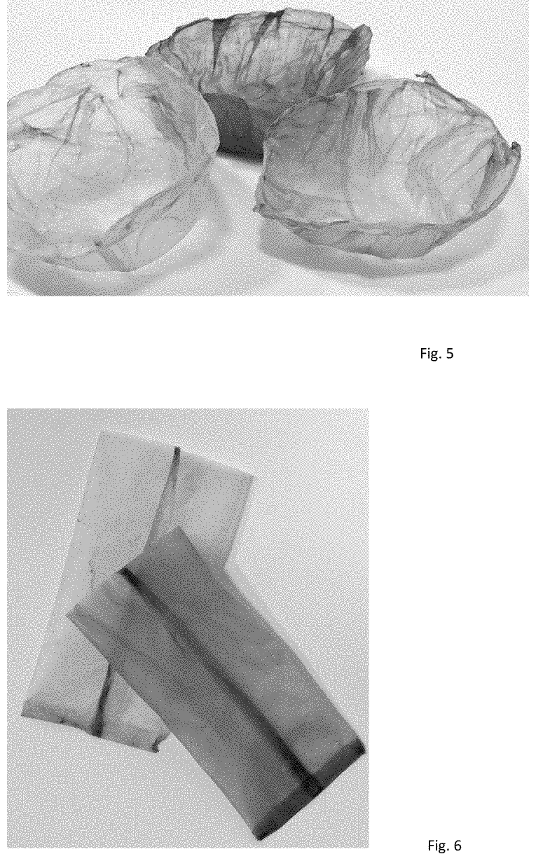

In reference to FIG. 5, it is shown a disposable tableware made of the raw material produced from industrial waste according to the disclosed method.

In reference to FIG. 6, artificial leather is shown.

According to the method of this invention, the use of biomass is a response to the increasing pollution of the natural environment. Moreover, it is an alternative to the production of plastic products, all the more that the utility materials made of it are not inferior to their durability, visual qualities, and usability than objects made of synthetic or semi-synthetic materials. Notably, the processed biomass is also characterized by purity, mechanical strength, the ability to absorb liquids, biocompatibility with living tissue, flexibility, and the ability to immobilize various substances with bioactive properties.

Typically, the sheets obtained by the method described here are translucent to transparent and with higher opacity when thickness is higher than 0.5 mm. The sheet's flexibility varies from very flexible to stiff, similarly to the flexibility of paper depending on the thickness and glycerin concentration or manufacturing methods—drying on substrate or pressing. Dry sheets in a form of foil are characterized by thickness from 6 to 1000 μm, preferably 50-200 μm, more preferably 50-150 μm. In certain aspects thickness of 100-125 μm is preferable. In certain aspects when forming artificial leather, the thickness of the dry sheets may be 300-20000 μm. Strength from of the sheets can be 0.5 MPA to 220 MPA, but not limited to this but depends on thickness, glycerin soaking parameters, manufacture method, suspension particle size. The dry sheets have a very high air barrier property which is a main advantage for usage of the dry sheets as a packaging material.

The method of producing sheets resulting from the fermentation of biomass, intended for the production of utility materials, will be used in industrial production. The invention is an effective alternative to the existing technologies to produce utility materials with similar applications, especially single-use, made of plastics, textiles, decorative materials, and others, as a coating specifically for paper or bioplastics or additive to other materials, specifically paper but not limited to it.

The invention is now described by way of non-limiting examples:

Example 1. Yield of the raw material is affected by the source of the waste source, pH, and additives.

Table 1 illustrates wet mass and dry mass yield of the raw material with different starting material and variables. The inoculum used in all these experiments was 10% of Komagataeibacter rhaeticus.

| | | | | | | | | |
|---|---|---|---|---|---|---|---|---|
| Red | 10 | 100 ml | — | 8 | 6.3 | 10 ml | 11.856 | 1.098 |
| beetroot | | | | | | | | |
| Coffee | 10 | 100 ml | — | 8 | 6.3 | 10 ml | 10.760 | 1.053 |
| husk | | | | | | | | |
| Apple | 10 | 100 ml | — | 8 | 6.3 | 10 ml | 10.006 | 1.004 |
| waste | | | | | | | | |
| Apple | 10 | 100 ml | 0.015 g | 8 | 6.3 | 10 ml | 19.303 | 1.951 |
| waste | | | CaCl2 | | | | | |
| Apple | 10 | 100 ml | — | 8 | 4 | 10 ml | 24.896 | 2.263 |
| waste | | | | | | | | |

Example 2 Pilot scale experiment produced in 4 fermentation batches 4200 kg of raw material per 4500 l of medium

A pilot scale experiment was conducted in a continuous process according to this invention. The experiment included 4 batches of fermentation and the total yield of raw material at the end of the experiment was 4200 kg, while the total amount of mixture in the containers was 4500 l. The inoculum used in this experiment was mix of a symbiotic culture of bacteria and yeast.

Ist Batch

Medium 1000 L/Ino 500 L=1000 containers 1.5 L

Harvest per container: 4 days, weight: ˜500 g/1.5 L

1000 containers/1500 L/Weight total 500 kg

Leftover fluid ˜1000 L

Cost 1500 L waste based medium

Product harvested: 500 kg raw material

II Batch

Reused fluid ˜500 L+1000 L fresh medium

Additive 400 L to 1000 Containers after 4 days, 1.9 L in container

Harvest per container 8 days

1000 containers/1900 L/Weight total ˜1000 kg

Leftover fluid ˜1000 L

Cost: 1000 L waste based medium

Product harvested: 1000 kg raw material

Batch 3

Reused fluid ˜500 L+1000 L fresh medium

Additive 400 L to 1000 Containers after 4 days, 1.9 L in container

Harvest per container 8 days

1000 containers/1900 L/Weight total ˜1300 kg

Leftover fluid ˜700 L

Cost: 1000 L waste based medium

Product harvested: 1300 kg raw material

Batch 4

Reused fluid ˜500 L+1000 L fresh medium

Additive 300 L to 1000 Containers after 4 days, 1.8 L in container

Harvest per container 8 days

1000 containers/1800 L/Weight total ˜1500 kg

Discarded fluid ˜300 L

Cost: 1000 L waste based medium

Product harvested: 1500 kg raw material

After batch 4 the process continues or starts from the beginning.

Total cost in 4 batches: 4500 L medium

Total Product harvested in 4 batches: 4200 kg of raw material

Example 3 illustrates the method as shown in FIG. 1. The element numbers of FIG. 1 are shown here

As shown in FIG. 1, the biomass 1 is filtered using filter material X1 having a size of 1 mm, and then the filtered biomass 1 is sent to mixing kettle (vessel) A. In mixing boiler A, the process of mixing primary material 1 is carried out simultaneously with the following: components in the following proportions: sulfuric acid ammonium salt (NH4) 2SO4 2 in the amount of 5% and constituting isolated microorganisms, producing cellulose or microorganisms in symbiosis with yeast, being in the form of a suspension inoculum 3 in the amount of 45% and so on the solution 4 obtained by mixing in the mixing kettle (vessel) A is subjected to a pH meter Y1 to check the pH value. Depending on the pH value, acetic acid CH3COOH 5 is added to solution (supplemented raw material) 4, then in solution 4 mixed with acetic acid CH3COOH 5 is checked with a refractometer Y2 for the sugar level. Solution 4 is filled with liquid glucose 6 until the sugar level in the amount of 5%. Solution 4, supplemented with liquid glucose 6, is left for 7 days, and then the resulting main mixture 7 prepared after previous activities in the mixing kettle A is poured into containers B. Part of the main mixture 7 is left in the mixing kettle A, and then the main mixture 7 poured into the containers B is subjected to an automatic incubation process. Throughout the incubation process, the main mixture 7 poured into containers B is supplemented with the main mixture left in the mixing kettle A until the desired level is equalized, and then in containers B from the main mixture 7, the preliminary material 8 is drained from the main mixture 8, which then it goes from the containers B to the container C. The liquid residue 9 remaining in containers B resulting from draining the preliminary raw material 8 is directed through pipe D to boiler E. At the same time, the containers B are emptied of preliminary raw material 8 and liquid residue 9 and are directed to the target purification at a temperature of 55° C. water 10, then the preliminary raw material 8 located in the container C is subjected to the process of cleaning with a sodium hydroxide solution of NaOH 11 in the amount of 1%, and then the purified raw material is subjected to the process of soaking with glycerin solution C 3 H 5 (OH) 12 in an amount of 15% over a period of 90 minutes. Further, soaked with glycerin solution C 3 H 5 (OH) 12, purified raw material is dried at 30° C. for 5 days on substrate F. After completion of the drying process, the main raw material 13 formed on substrates 13 is separated is left from the substrate F and cut into dry sheets 14.

FIGS. 2, 3, and 4 show a process flow chart showing the method of producing specific usable materials from the sheets resulting from the fermentation of the biomass after the entire process according to the invention has been carried out.

Example 4 illustrates the method to make disposable vessels as shown in FIG. 2. Element numbers refer to FIG. 2.

As indicated in FIG. 2, dry sheet 14 is moistened 15 to a minimum of 10% water content, after which the blank 16a thus formed is placed in a press 17 with a pressure of 25 tons at 125° C. As a result, a disposable vessel 20 of the desired shape is removed from the press. FIG. 5 shows examples of the vessels.

Example 5 illustrates the method to make sachets as shown in FIG. 3. Element numbers refer to FIG. 3.

FIG. 3 shows dry sheet 14, which is subjected to moisture 15 until reaching a water content of 10%, after which the blank 16b thus formed is placed in a foil sealing device 18. In the final phase, a sachet 21 of the desired shape is removed from the foil sealing device. FIG. 6 shows examples of the sachets.

Example 6 illustrates the method to make artificial leather as shown in FIG. 4. Element numbers refer to FIG. 4.

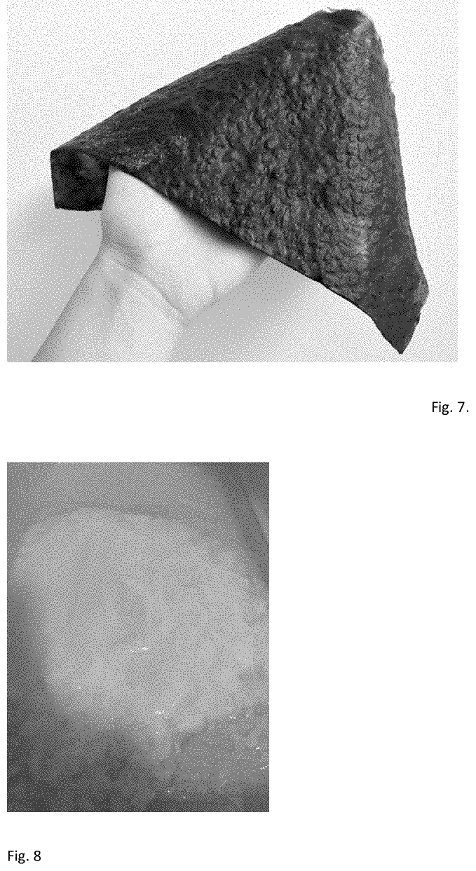

FIG. 4 is a view of a dry sheet 14 that has been placed in a press 17, in which a pressure of 5 tons is applied at a temperature of 125° C., and then the resulting blank 16c is impregnated with a mixture of guar gum and natural resins 19. After completion, impregnation, artificial leather 22 is obtained. FIG. 7 shows an example of the artificial leather.

Example 7. Sheet suspension-additive product for flexible materials composites

Preliminary raw material is blended for 5 min, there is an option that water is added to allow blending of dry sheets into suspension from 0.1 to 10% of fiber content. The blended preliminary raw material is cleaned with 1% NAOH solution for 60 min in 60° C. The suspension is later rinsed with water to obtain neutral pH. This suspension is blended again to avoid uneven particles in suspension. The suspension is ready to be added in the paper making process, as a coating for bioplastics and ingredients to other materials but not limited to it. FIG. 8 shows an example of suspension.

Reference Example 8 Material sheets method from slurry

The suspension was prepared like in reference example 7.

Suspension is divided on half to conduct two drying methods

Suspension is spread by roller, casting knife or spray nozzles on Teflon coated substrate and 2 mm thickness sheet was formed from them and subjected to various drying methods—on a heating plate at 55 degrees and a dryer. After drying the formed dried sheets are separated from substrate and cut.

Example 9 Using suspension of this invention as paper additive.

The table 2 below shows how the suspension when added into paper pulp containing softwood and hardwood in relation of 20:80 notably decreases air permeability. The suspension was made as in example 7 above. Either 5 or 10% or the suspension was added to paper pulp. Air permeability increased markedly. With these characteristics the pulp is an excellent additive in paper making process especially when making paper for food preservation.

TABLE 2

| long(soft- | Suspension | | | |
|---|---|---|---|---|
| wood):short | from sheets | | | |
| (hardwood) = | described in | Thick- | | Air |
| 20:80 | invention | ness | Grammage | permability |
| 100% | 0% | 125.1 | 80.3 | 1280 |
| | | μm | g/m2 | mL/min |
| 95% | 5% | 121.4 | 79.7 | 220 |
| 90% | 10% | 117.6 | 80.0 | 110 |

Reference Example 10 Inoculum characterization

Preferably inoculum is characterized by containing SCOBY(Symbiotic culture of bacteria of Yeast) or SCOBY and KR in proportion 29:1, 25:5, 1:1 preferably 25:5 or mix of cellulosic bacteria like Komagataeibacter rhaeticus and or Komagataeibacter xylinus and Saccharomyces cerevisiae yeasts, with bacteria cells exceeding yeast cells (population higher than 50%).

What is claimed is:

1. A method to produce cellulose containing biodegradable materials from household or industry waste, the method comprising:
a. obtaining biomass derived from the waste;
b. mixing the biomass with water and raising the temperature to at least 120° C.;
c. obtaining a filtered primary raw material by filtering the biomass;
d. directing the filtered primary raw material into one or more vessels;
e. supplementing the filtered primary raw material in the one or more vessels with one or more of ammonium salt of sulfuric acid, calcium, and magnesium; and adding an inoculum suspension comprising cellulose producing bacteria;
f. adjusting pH of the supplemented primary raw material in the one or more vessels to below pH 7, and sugar level of the material to 5-15 w-%;
g. obtaining a main mixture by allowing the supplemented primary raw material from step f) to ferment in the one or more vessels at 22-33° C.;
h. dividing part of the main mixture into containers and incubating the main mixture in the containers at 22-33° C.;
i. adding portions of the main mixture incrementally from the one or more vessels to the containers every 4-15 days throughout the incubation period of step h);
j. Obtaining a raw material from the containers by draining the main mixture form the containers and collecting the raw material into a collection container and directing the drained liquid back to the one or more vessels;
k. obtaining purified raw material by cleaning the raw material in the collection container; and
l. drying the purified raw material on a substrate to form dry cellulosic sheets.

2. The method of claim 1, wherein the cellulose producing bacteria is selected from the group consisting of Acetobacter sp, Glucobacter sp, Sarcina sp, and Komagataeibacter sp.

3. The method of claim 1, wherein the inoculum suspension comprises additionally yeast cells.

4. The method of claim 3, wherein the inoculum comprises more bacterial cells than yeast cells.

5. The method of claim 3, wherein the inoculum suspension comprises a symbiotic culture of bacteria and yeast, or Saccharomyces cerevisiae cells.

6. The method of claim 1, wherein the temperature is raised in step a) to at least 120° C. for 15 to 120 minutes.

7. The method of claim 1, wherein in step e) the filtered primary raw material is supplemented with calcium in an amount of 0.005-1.0 g/l.

8. The method of claim 1, wherein efficiency of production of bacterial cellulose is 50-950 g/l of the main mixture.

9. The method of claim 1, wherein efficiency of production of dry sheets is 5-90 g/l of the main mixture.

10. The method of claim 1, wherein the method further comprises a step of blending the raw material or the purified raw material to obtain a suspension.

11. The method of claim 10, wherein the suspension obtained is supplemented with grinded dry sheet material.

12. The method of claim 1, wherein the method further comprises a step of treating the purified raw material with glycerin.

13. The method of claim 1, wherein the dry sheets are further treated to form sachets, disposable vessels, or artificial leather.

14. The method of claim 13, wherein the dry sheet are treated to form disposable vessels and method comprises a step of moistening the dry sheets to a minimum of 10% water content and placing the moistened sheet in press with a pressure of at least 25 ton at a temperature up to 125° C.

15. The method of claim 13, wherein the dry sheets are treated to form sachets and the method comprises a step of moistening the dry sheets to a minimum of 10% water content and placing the moistened sheet in a foil welding device.

16. The method of claim 1, wherein the dry sheets are treated to form artificial leather and the method comprises a step of moistening the dry sheets to a minimum of 10% water content and placing the moistened sheet in in press with a pressure of at least 5 tons at a temperature of up to 125° C. and impregnating the pressed material with a mixture of guar gum and natural resins.

17. A cellulosic dry sheet produced by the method of claim 1.

18. The cellulosic dry sheet of claim 14, wherein the thickness of the sheet is between 6 and 20 000 μm.

19. A cellulosic suspension produced by the method of claim 10.

20. The cellulosic suspension of claim 16, suitable to be added as an additive to paper.
